(19) United States
(12) Patent Application Publication
(10) Pub. No.: US 2019/0269366 A1
(43) Pub. Date: Sep. 5, 2019

(54) Device for Acquiring Brain Activity Signals

(71) Applicant: URGOTECH, Paris (FR)

(72) Inventors: Philippe Lantenois, Oullins (FR); Joseph Mazoyer, Ste Foy Les Lyon (FR)

(73) Assignee: URGOTECH, Paris (FR)

(21) Appl. No.: 16/345,459

(22) PCT Filed: Oct. 27, 2017
(86) PCT No.: PCT/EP2017/077594

§ 371 Date: Apr. 26, 2019

(30) Foreign Application Priority Data
Oct. 28, 2016 (FR) 1660503

(51) Int. Cl. A61B 5/00; A61B 5/0478

(52) U.S. Cl. CPC A61B 5/6803; A61B 5/0478

(57) ABSTRACT

The present disclosure relates to an acquisition device intended to be positioned on the head of a user in order to perform an encephalogram. The device comprises at least one acquisition electrode (32, 34, 33) configured in order to be positioned in contact with the head of the user at at least any one of the positions of the international 10-20 system, so as to acquire at least one brain activity signal representative of the brain waves of the user and at least one first and one second means of adjusting the position of the acquisition electrode in a first and a second plane, respectively.

### FIELD OF THE DISCLOSURE

The present invention relates to a device for acquiring an encephalogram, intended to be positioned on the head of a user so as to acquire a brain activity signal, in particular in order to allow the characterization and/or treatment of sleep, hyperactivity disorders, migraine-related disorders, memory disorders, cognitive disorders, etc. of the user. The invention also relates to a method for positioning such an acquisition device on the head of a user, optionally operated by the user himself.

### BACKGROUND OF THE DISCLOSURE

Thus, in order to characterize in particular the sleep of an individual, it is known to identify “sleep spindles”. The sleep spindles are electrical brain activity signals with frequencies generally comprised between 9 and 16 Hz (Molle et al., 2011) and amplitude ranging from 25 to 150 micro volts. Low-frequency and high-frequency sleep spindles are found, which are variable and specific to each individual. The sleep spindles generally last between 0.5 and 2 seconds and are the product of an activity of the reticulo-thalamo-cortical system. It has been demonstrated that producing a high density of sleep spindles is associated with effective sleep.

Identification of the sleep spindles is possible by acquiring brain activity signals at specific positions on the head of the user, in particular at positions C3, C4 and/or Cz defined by the international 10-20 system (see in particular document WO-A1-2009/061920).

The international 10-20 system is mentioned in particular in patent application WO-A1-2009/061920. This system, shown in FIG. 1 (according to the modified combinatorial nomenclature guidelines No. 5: Guidelines for Standard Electrode Position Nomenclature, American Clinical Neurophysiology Society, 2006), is after all an internationally recognized method for locating the possible positioning of at least one electrode on the surface of a human cranium in the context of performing an electroencephalogram. In this system, each electrode is thus identified by a letter coding the position thereof with respect to the major regions of the brain, and a number, or the letter z, which defines the hemisphere (Jasper, 1958):
the letters F, T, C, P and O indicate respectively the Frontal, Temporal, Central, Parietal and Occipital regions,
the even numbers (2, 4, 6, 8) correspond to the right hemisphere,
the odd numbers (1, 3, 5, 7) correspond to the left hemisphere,
the letter z indicates the electrodes situated on the midline.

FIG. 1 thus shows the different positions of this international 10-20 system.

Another potential application relates to attention deficit disorder, with or without hyperactivity (ADHD). In this application, acquiring brain activity signals can equally well take place at positions C3, C4 and/or Cz (optionally and/or CPz and/or FCz) of the international 10-20 system.

With regard to yet another application, this relates to memory disorders, in particular within the context of Alzheimer's disease. In this application, acquiring brain activity signals itself can be performed at positions C3, C4, T7 and/or T8 of the international 10-20 system, the positions T7 and T8 corresponding respectively to the positions T3 and T4 according to a previous simplified nomenclature of this international 10-20 system. A variant seriously considered may consist of acquiring these brain activity signals at positions C3, C4 CPz and/or FT7 of the international 10-20 system.

It is also known to perform the acquisition of the brain activity signals by arranging a headset equipped with a plurality of electrodes on the head of a user. By way of example, document U.S. Pat. No. 4,967,038 B concerns a cap on which electrodes are mounted. The cap is produced in stretchable fabric such that it is able to adapt to heads of different sizes and shapes.

However, a major drawback of this cap is that the electrodes are poorly positioned against the scalp of the user, as they have a tendency to pivot on themselves, making the acquisition unsatisfactory.

Another example of an acquisition headset is described in document WO-A1-2009/045407, in which the headset comprises an armature formed by inextensible bands tightly fitting around the head of the user. The electrodes are mounted on these inextensible bands. In order to allow the headset to adapt to different head geometries, extensible bands are arranged between certain inextensible bands in order to make it possible to distance or separate the inextensible bands with respect to one another. In addition, elastic elements are arranged between the electrodes in order to hold the electrodes securely against the scalp of the user.

However, the main drawback of this headset is to allow only overall adjustment of the headset with respect to the head geometry of the user. Indeed, the extensible bands provide localized degrees of freedom allowing the headset to adapt to the head of the user and thus to modify the distance between the electrodes. However, the headset does not make it possible to know and quantify the specific position of each of the electrodes. As a result, subsequent use of the headset on one and the same user does not guarantee that the electrodes are placed at a position that is suitable for the morphology of the user. The inability to arrange the electrodes in a predetermined position suitable for the user increases the risks of poor positioning of the electrodes, in particular in the event that the subject seeks to arrange said electrodes alone, without outside help. In addition, positioning the headset is complex and time -consuming.

Finally, document WO-A1-2015/153278 also concerns a headset for collecting brain activity signals comprising a plurality of support bands intended to extend around the head of the user in order to tightly fit around it and a plurality of electrodes mounted on the support bands. The headset also comprises a series of lines extending between several of the support bands and slidingly passing through one or more of the electrodes. Adjustment elements are linked to the lines in order to make it possible to reduce or increase the distance separating the support bands so as to hold the electrodes securely against the head of the user. The support bands and the lines can be elastic in order to improve the secure hold of the electrodes against the head of the user.

However, this headset has the same drawbacks as the headset described previously, as the adaptation to the morphology of the head of the user is carried out globally, without allowing the possibility of a specific and quantifiable adjustment of the position of the electrodes.

### SUMMARY OF THE DISCLOSURE

There is therefore a need for a device for acquiring brain activity signals with improved positioning of the electrodes on the head of a user, in particular by making it possible to facilitate the positioning of the electrodes in a predetermined position on the head of users having different morphologies.

To this end, the present invention proposes an acquisition device intended to be positioned on the head of a user in order to perform an encephalogram, the head of the user being defined in a transverse plane, a sagittal plane and a frontal plane, the device comprising:
at least one acquisition electrode configured in order to be positioned in contact with the head of the user at at least one of the positions of the international 10-20 system, so as to acquire at least one brain activity signal representative of the brain waves of the user;
at least one first and one second means of adjusting the position of the acquisition electrode in a first and a second plane, respectively;
   in which the first and second planes are different and chosen from at least the transverse and sagittal planes of the head of the user,
in which said first and second means of adjusting the position of the acquisition electrode are capable of being actuated independently of one another.

According to an embodiment of the device, the latter also comprises:
a posterior support;
an anterior support capable of engaging with the posterior support in order to form an annular portion intended to tightly fit around the head of the user in the transverse plane and/or capable of engaging with the posterior support in order to form an arched portion intended to tightly fit around the head of the user in the sagittal plane,
an upper support on which is mounted the acquisition electrode, the upper support being mounted on the anterior or posterior support.

According to another embodiment of the device, the latter also comprises a third means of adjusting the position of the acquisition electrode in a third plane chosen from the transverse, sagittal and frontal planes of the head of the user, the first, second and third planes being different, the first, second and third means of adjusting the position of the acquisition electrode preferably being capable of actuation independently of one another.

According to another embodiment of the device, the upper support is combined with the posterior support, in which:
the first adjustment means is capable of displacing the acquisition electrode with respect to the upper support in a first direction comprised within the frontal plane;
the second adjustment means is capable of displacing the anterior support with respect to the posterior support in a second direction comprised within the sagittal plane; and
the third adjustment means is capable of displacing the anterior support with respect to the posterior support in a third direction comprised within the transverse plane.

According to another embodiment of the device, the first adjustment means comprises a slider capable of sliding in the first direction comprised within the frontal plane in a frontal housing formed in the upper support, the at least one acquisition electrode being mounted on the slider.

According to another embodiment of the device, the second and third adjustment means comprise:
a rod formed on the anterior support;
a housing formed in the posterior support in which the rod is capable of sliding; and
a direct or indirect means of adjusting the position of the rod mounted on the posterior support and capable of cooperating with the rod in order to slide the rod inside the housing so as to displace the anterior support with respect to the posterior support.

According to another embodiment of the device, the second and third adjustment means comprise at least one projection and at least one recess formed on the rod or the direct or indirect adjustment means, respectively, the projection and the recess being capable of cooperating with one another in order to hold the rod in a predetermined position with respect to the housing.

According to another embodiment of the device, the device comprises a first, and/or a second and/or a third acquisition electrodes, configured in order to be positioned in contact with the head of the user at one of the positions C3, C4 and/or Cz of the international 10-20 system, respectively, and in which the first, and/or second and/or third acquisition electrodes are mounted on the upper support.

The device can also comprise an acquisition electrode configured in order to be positioned in contact with the head of the user at the position CPz of the international 10-20 system, and/or an acquisition electrode configured in order to be positioned in contact with the head of the user at the position FT7 of the international 10-20 system.

Alternatively, the device can also comprise an acquisition electrode configured in order to be positioned in contact with the head of the user at the position T7 of the international 10-20 system, and/or an acquisition electrode configured in order to be positioned in contact with the head of the user at the position T8 of the international 10-20 system.

According to another embodiment of the device, the anterior support comprises a bearing portion configured in order to bear on an ear of the user when the acquisition device is arranged on the head of the user.

According to another embodiment of the device, the bearing portion comprises at least one reference electrode configured in order to be positioned in contact with the head of the user, preferably facing one of the mastoid processes of the user.

According to another embodiment of the device, the upper support is mounted on the posterior support, the posterior support comprising a hollow capable of indicating a reference position of the posterior support, the hollow preferably being configured in order to be arranged facing the external occipital protuberance, preferably at the inion, of the head of the user.

According to another embodiment, the device also comprises:
a processing unit connected to the at least one acquisition electrode, and, if appropriate, to the at least one reference electrode in order to process information transmitted by the at least one acquisition electrode and the at least one reference electrode;
a unit for the transmission of the information processed by the processing unit.

In addition, the invention also relates to a method for positioning an acquisition device on the head of a user in order to perform an encephalogram, the head of the user being defined in a transverse plane, a sagittal plane and a frontal plane, the method comprising the steps consisting of:
Providing an acquisition device as described above;
arranging the acquisition device on the head of the user;
actuating the third adjustment means in order to tightly fit around the head of the user,
actuating the second adjustment means so as to place the upper support equidistant from a distal end of the anterior transverse portion and from a distal end of the posterior transverse portion,
actuating the first adjustment means in order to adjust the position of the at least one acquisition electrode in the frontal plane;
   in which the steps of actuating the first and second adjustment means are performed independently.

According to an embodiment of the method, the latter also comprises the steps consisting of:
determining a first and a second adjustment positions of the first and second adjustment means, respectively;
memorizing the first and second adjustment positions;
actuating the first and second adjustment means so as to position them in adjustment positions different from the first and second adjustment positions;
removing the acquisition device from the head of the user;
replacing the acquisition device on the head of the user;
actuating the first and second adjustment means so as to arrange them in the first and second adjustment positions.

According to another embodiment of the method, the acquisition device is an acquisition device as described above, in which the step consisting of arranging the acquisition device on the head of the user comprises the steps consisting of:
displacing the anterior support with respect to the posterior support so that the annular portion has a circumference equal to or greater than the maximum circumference of the head of the user, considered in the transverse plane;
positioning the acquisition device on the head of the user so that the bearing portion bears on an ear of the user;
positioning the acquisition device on the head of the user so that the recess of the posterior support is facing the external occipital protuberance of the head of the user.

### BRIEF DESCRIPTION OF THE DRAWINGS

Other characteristics and advantages of the invention will become apparent on reading the following description of a preferred embodiment of the invention, given by way of example with reference to the attached drawings.

FIG. 1 already mentioned above shows diagrammatically the positions in the international 10-20 system. FIGS. 2 to 4 show diagrammatically several perspective views of an example device for acquiring brain activity signals arranged on the head of a user, within the context of studying sleep disorders of this user.

FIG. 5 shows a diagram illustrating a method for characterizing sleep, implemented by the acquisition device in FIGS. 2 to 4.

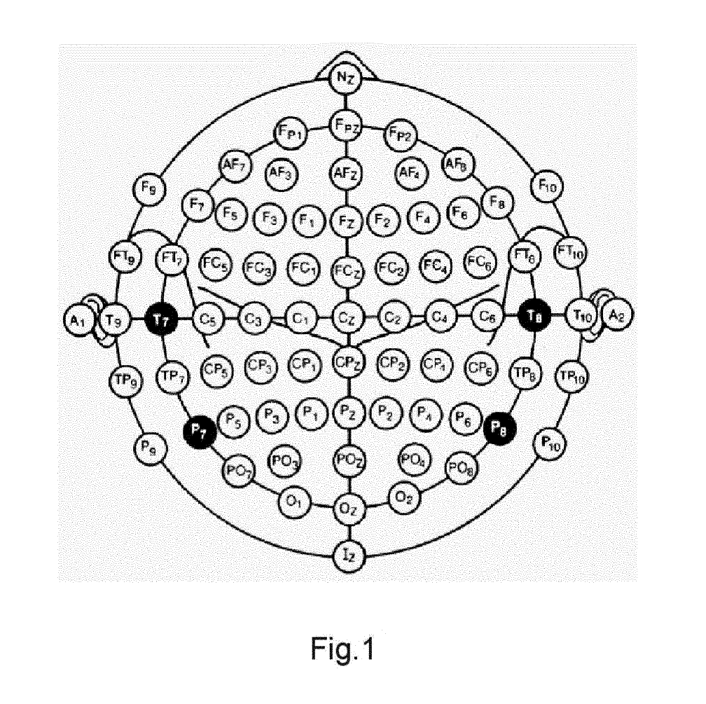

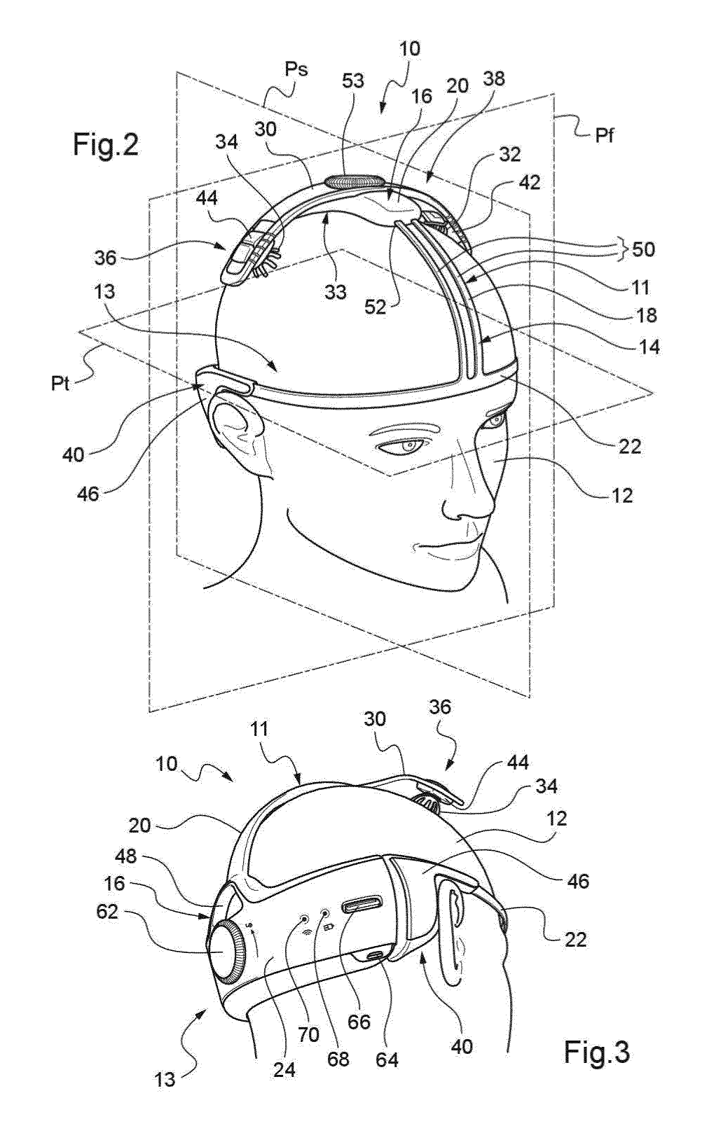

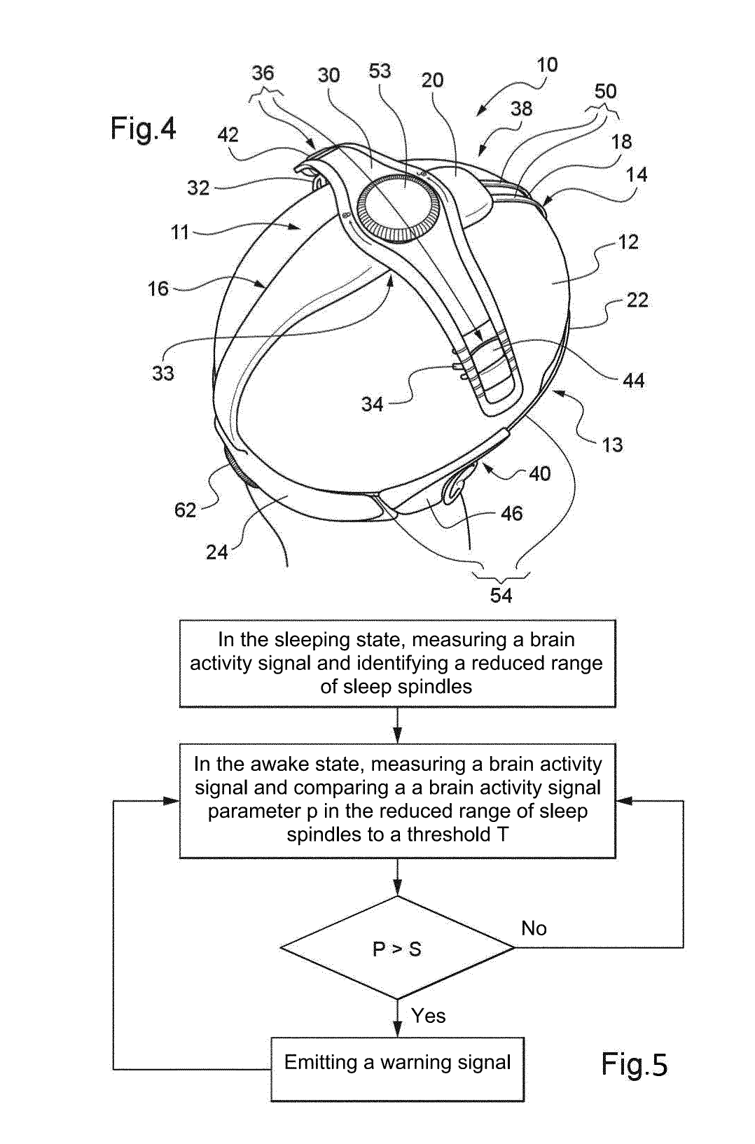

### DETAILED DESCRIPTION

With reference to FIG. 2, an acquisition device 10 is proposed, intended to be arranged on the head of a user 12, in order to acquire brain activity signals representative of the brain waves of the user 12. These brain activity signals make it possible in particular to produce an encephalogram. The head of the user 12 is defined in a transverse plane Pt, a sagittal plane Ps and a frontal plane Pf, forming an orthonomic reference frame. Alternatively, it is also possible to define the head of the user 12 by any combination of planes or any reference frame.

The acquisition device 10 is intended to acquire brain activity signals making it possible subsequently, among other things, to identify sleep spindles, or other desired waves. To this end, the acquisition device 10 makes it possible to acquire all of the brain activity signals that can be captured facing the placement of the at least one of the electrodes at C3, C4 or Cz, so as to manage to collect, via filters, only the signals having a frequency for example comprised between 9 and 16 Hz, an amplitude greater than 15 μV and a period comprised between 0.5 s and 2 s. It is of course possible in the same way to collect other signals having frequencies, amplitudes and periods other than those described above, in particular in order to characterize any hyperactivity disorders or those associated with migraine in a user. The acquisition device 10 can thus be used as a device or system for characterizing sleep as described in patent application FR 1556421 (from Urgotech) or in a method for characterizing sleep also proposed hereinafter in this document.

The acquisition device 10 is in the form of a headset capable of resting and being held on the head of the user 12. The acquisition device 10 extends above the head of the user 12 in the sagittal plane Ps in the form of an arched portion 11 and around the head of the user 12 in the form of an annular portion 13, or crown, in order to tightly fit around the head of the user 12. The arched portion 11 is connected at its ends to the annular portion 13 in order to be able to rest the acquisition device 10 on the head of the user 12.

As an alternative to the annular portion 13 and the arched portion 11, any other geometry making it possible to hold the acquisition device 10 on the head of the user 12 can be used. In particular, the acquisition device 10 can comprise one or more arched portions 11 extending in a plane that is parallel or angularly offset with respect to the sagittal plane Ps. In addition, the annular portion 13 can be discontinuous or partially surround the head of the user 12 while making it possible to hold the acquisition device 10 on the head of the user 12. Similarly, the arched portion 11 can be discontinuous or partially surround the head of the user 12.

In order to allow the acquisition of the brain activity signals, the acquisition device 10 can comprise at least one of a first acquisition electrode 32, a second acquisition electrode 34 or a third acquisition electrode 33 intended to be positioned at positions C3, C4 and Cz, respectively, of the international 10-20 system. In these positions C3, C4, and Cz, the first 32, second 34 and third 33 acquisition electrodes are suitable, alone or in combination, for acquiring brain activity signals making it possible in particular to identify sleep spindles. In order to facilitate the positioning of the first 32, second 34 and third 33 acquisition electrodes, the latter are mounted on the arched portion 11 surrounding the top of the head of the user 12. Alternatively, the acquisition device 10 can be provided with only a single acquisition electrode chosen from the first 32, second 34 and third 33 acquisition electrodes.

In addition, the acquisition device 10 can comprise a reference electrode making it possible to carry out a reference measurement with respect to the brain activity signals acquired with the first 32, second 34 and/or third 33 acquisition electrodes. This reference electrode is preferably positioned facing one of the mastoid processes of the user 12. In addition, the acquisition device 10 can comprise a passive electrode (not shown) making it possible to ground the acquisition device 10. This passive electrode is preferably also arranged facing one of the mastoid processes of the user 12, for example facing the other mastoid process than the one where the reference electrode is arranged. Alternatively, the first 32, second 34 and/or third 33 acquisition electrodes can also combine a reference and/or ground function in order to allow the acquisition device 10 to dispense with the reference electrode and/or the ground electrode.

The first 32, second 34 and third 33 acquisition electrodes, the passive electrode and the reference electrode can be dry, wet, semi-dry electrodes or also semi-wet electrodes. Each type of electrode can have a particular and different nature.

In order to ensure satisfactory positioning of the first 32, second 34 and third 33 acquisition electrodes at positions C3, C4 and Cz for users 12 having different head morphologies, the acquisition device 10 comprises a first (36, 38, 40) and a second (36, 38, 40) means of adjusting the position of the first 32, second 34 and third 33 acquisition electrodes. Thus, by allowing an adjustable positioning of the first 32, second 34 and third 33 acquisition electrodes, the acquisition device 10 ensures satisfactory acquisition of the brain activity signals for users 12 having a different head morphology. This is the case for example when the users have different ages.

In order to allow a bi-axial positioning of the first 32, second 34 and third 33 acquisition electrodes, the first (36, 38, 40) and second (36, 38, 40) adjustment means allow the adjustment of the first 32, second 34 and third 33 acquisition electrodes in a first and a second different planes chosen from at least the transverse Pt and sagittal Ps planes of the user 12. Thus, the advantages associated with the positioning of the first 32, second 34 and third 33 acquisition electrodes are improved, since the acquisition device 10 can ensure a satisfactory acquisition of the brain activity signals for users 12 having different head morphologies.

In addition, in order to simplify the positioning of the first 32, second 34 and third 33 acquisition electrodes, the first (36, 38, 40) and second (36, 38, 40) adjustment means can be actuated independently of one another. The independent actuation of the first (36, 38, 40) and second (36, 38, 40) adjustment means makes it possible to separate the movement of the first 32, second 34 and third 33 acquisition electrodes associated with the first (36, 38, 40) and second (36, 38, 40) adjustment means. Thus, in combination with the fact that the first (36, 38, 40) and second (36, 38, 40) adjustment means allow an adjustment in different planes, separating the displacement of the first 32, second 34 and third 33 acquisition electrodes is possible in two planes from the frontal Pf, transverse Pt and sagittal Ps planes. In other words, it is possible to selectively displace the first 32, second 34 and third 33 acquisition electrodes in one from the transverse Pt, sagittal Ps and frontal Pf planes. In addition, this separation makes it possible to obtain a targeted, more accurate positioning of the first 32, second 34 and third 33 acquisition electrodes than in an acquisition device in which the displacements of the acquisition electrodes are combined, as for example in document WO-A1-2015/153278. In addition, separation of the positioning of the first 32, second 34 and third 33 acquisition electrodes allows quantification of the position of the first 32, second 34 and third 33 acquisition electrodes in two of the frontal Pf, transverse Pt and sagittal Ps planes, thus allowing simple and rapid repeatability of the first 32, second 34 and third 33 acquisition electrodes.

In order to obtain a satisfactory compromise between the simplicity of the acquisition device 10 and the satisfactory positioning of the first 32, second 34 and third 33 acquisition electrodes, the first plane in which the first adjustment means 36 is capable of adjusting the position of the first 32 and second 34 acquisition electrodes is the frontal plane H. The second plane in which the first adjustment means 36 is capable of adjusting the position of the first 32 and second 34 acquisition electrodes is chosen from the transverse Pf and sagittal Ps planes. In this way, the acquisition device 10 is capable of positioning, in particular with the help of the second adjustment means 38, the first 32 and second 34 acquisition electrodes so as to align them in the frontal plane Pf. The first adjustment means 36 then allows the adjustment of the position of the first 32 and second 34 acquisition electrodes in the frontal plane Pf. In this case, the acquisition device 10 can be used so that the acquisition device 10 is arranged on the head of the user 12. The second adjustment means 38 is actuated in order to grip the head of the user 12. Then, the first adjustment means 36 is actuated in order to adjust the position of the first 32 and second 34 acquisition electrodes in the frontal plane Pf.

In order to allow the acquisition device 10 to adapt to a greater range of different morphologies and ages, the acquisition device 10 can comprise a third adjustment means 40 of the position of the first 32 and second 34 acquisition electrodes in a third plane different from the first and second planes. In this case, the position of the first 32 and second 34 acquisition electrodes can be adjusted in each of the frontal Pf, transverse Pt and sagittal Ps planes. In addition, the first 36, second 38 and third 40 adjustment means can be actuated independently in order to allow separate movements of the first 32 and second 34 acquisition electrodes, and thus to simplify the positioning thereof. The addition of a third adjustment means 40 makes it possible to obtain the advantages associated with the first and second adjustment means mentioned above in an improved manner. By way of example, the acquisition device 10 presented in FIGS. 2 and 4 and comprising the first 36, second 38 and third 40 adjustment means is capable of adaptation to morphologies ranging from a 6-year-old user to an adult user.

In order to facilitate the repeatability of the positioning of the first 32, second 34 and third 33 acquisition electrodes, the acquisition device 10 is in the form of a rigid headset. By rigid is meant the fact that the headset is configured so that the position of the first 32, second 34 and third 33 acquisition electrodes with respect to the annular 13 and arched 11 portions can only be modified by actuating one or more of the first 36, second 38 and third 40 adjustment means, whether or not the headset is on the head of the user 12. The positioning of the first 32, second 34 and third 33 acquisition electrodes is therefore not dependent on whether the user 12 is wearing the headset. The rigidity of the headset thus makes it possible to retain, partially or totally, the positioning of the first 32, second 34 and third 33 acquisition electrodes in the sagittal Ps, transverse Pt and frontal Pf planes, even when the headset is not arranged on the head of the user 12. By way of comparison, the headset described in document WO -A1-2015/153278 cannot be considered to be a rigid headset within the meaning of the present description, as in this document, removal of the headset by the user necessarily loses the position of the electrodes, since the position of the electrodes is dependent on the headset being worn by the user.

In the embodiment described in FIGS. 2 to 4, the acquisition device 10 comprises an anterior support 14 and a posterior support 16 linked together so as to form the annular portion 13 and the arched portion 11 described above. In particular, the anterior support 14 comprises an anterior sagittal portion 18 extending in the sagittal plane Ps and re-joining a posterior sagittal portion 20 of the posterior support 16 in order to form the arched portion 11. Similarly, the anterior support 14 comprises an anterior transverse portion 22 linked to a posterior transverse portion 24 (see FIG. 3) of the posterior support 16 in order to form the annular portion 13.

The acquisition device 10 also comprises an upper support 30 associated with the arched portion 11. The upper support 30 extends in the frontal plane Pf on either side of the arched portion 11. In other words, the upper support 30 is associated with the arched portion 11 so as to be arranged on top of the head of the user 12. In particular, the upper support 30 is associated with the posterior support 16, at the level of the posterior sagittal portion 20. When the acquisition device 10 comprises the third acquisition electrode 33 intended to be positioned at the position Cz, this third acquisition electrode is under the upper support 30 so as to be facing the head of the user 12.

The embodiment of the acquisition device 10 presented in FIGS. 2 to 4 has a simple geometry along the three frontal Pf, transverse Pt and sagittal Ps planes of the head of the user 12. In fact, the acquisition device 10 is formed by an annular portion 13 extending in the transverse plane Pt, an arched portion 11 extending in the sagittal plane Ps and an upper support 30 extending in the frontal plane Pf. The configuration of the acquisition device 10 therefore facilitates integration of the adjustment means of the position of the first 32, second 34 and third 33 acquisition electrodes. Indeed, as a result of this geometry in the three frontal Pf, transverse Pt and sagittal Ps planes, it is possible to vary the position of the first 32, second 34 and third 33 acquisition electrodes by varying the dimensions or by displacing the upper support 30 in a direction comprised within the frontal plane Pf, the arched portion 11 in a direction comprised within the sagittal plane Ps and the annular portion 13 in a direction comprised within the transverse plane Pt.

The acquisition device 10 also comprises a first 42 and a second 44 sliders constituting all or part of the first adjustment means 36 of the first 32, second 34 and third 33 acquisition electrodes. The first 42 and second 44 sliders are mounted in a frontal housing on the upper support 30 on either side of the arched portion 11. The first 32, second 34 and third 33 acquisition electrodes are mounted on the first 42 and second 44 sliders so as to be positioned in contact with the head of the user 12 at the positions C3, C4 and Cz, respectively. In addition, the first 42 and second 44 sliders can be displaced with respect to the support 30 in a direction comprised within the frontal plane Pf so as to be able to adjust the position of the first 32, second 34 and third 33 acquisition electrodes in the frontal plane Pf. The displacement of the first 42 and second 44 sliders can be carried out manually. In order to allow a more accurate and quantifiable positioning, the first 42 and second 44 sliders can comprise projections capable of engaging with recesses formed in the support 30. Cooperation between the projections and the recesses makes it possible to place the first 42 and second 44 sliders, and thus the first 32, second 34 and third 33 acquisition electrodes in predetermined positions. Alternatively, the projections can be provided on the upper support 30 and the recesses on the first 42 and second 44 sliders. Also alternatively, it is possible to adapt any system allowing the first 32, second 34 and third 33 acquisition electrodes to adopt predetermined positions.

In order to adjust the position of the first 32, second 34 and third 33 acquisition electrodes in the sagittal plane Ps, the anterior 18 and posterior 20 sagittal portions are configured in order to be displaced with respect to one another in a direction comprised within the sagittal plane Ps. Displacement of the anterior 18 and posterior 20 sagittal portions with respect to one another constitutes the second adjustment means 38. Parallel to the positioning of the first 32, second 34 and third 33 acquisition electrodes in the sagittal plane Ps, the second adjustment means 38 also makes it possible to adapt the acquisition device 10 to the head of the user 12 in the sagittal plane Ps. In particular, the second adjustment means 38 comprises a sagittal rod 50 and a sagittal housing 52 formed in the posterior support 16, inside the posterior sagittal portion 20. The sagittal rod 50 is capable of sliding inside the sagittal housing 52 in order to displace the anterior 18 and posterior 20 sagittal portions with respect to one another. The sagittal rod 50 can be formed by two rod elements. In the embodiment of FIGS. 2 to 4, the sagittal rod 50 is merged with the anterior sagittal portion 18. Alternatively, the sagittal rod 50 can be formed projecting from the anterior sagittal portion 18. As an alternative to the sagittal rod 50 and the sagittal housing 52, the second adjustment means 38 can be formed by any system making it possible to displace the anterior 18 and posterior 20 sagittal portions with respect to one another.

In order to allow the adjustment of the position of the anterior 18 and posterior 20 sagittal portions with respect to one another, the second adjustment means 38 also comprises a means 53 for sagittal adjustment of the position of the sagittal rod 50. The sagittal adjustment means 53 is capable of engaging with the sagittal rod 50 in order to slide the sagittal rod 50 inside the sagittal housing 52 so as to displace the anterior support 14 with respect to the posterior support 16. The sagittal adjustment means 53 adopts the form of a button that can be displaced in rotation about itself in order to slide the sagittal rod 50 inside the sagittal housing 52. The turn button allows the second adjustment means 38 to be grasped easily. As an alternative to a turn button, the sagittal adjustment means 53 can be in any form making it possible to slide the sagittal rod 50 inside the sagittal housing 52. The adjustment means 53 is here described as a direct adjustment means.

In order to hold the sagittal rod 50 in a predetermined position with respect to the sagittal housing 52, the second adjustment means 38 can comprise at least one projection formed on the sagittal rod 52 and at least one recess formed on the sagittal adjustment means 53. The projection and the recess are capable of engaging with one another in order to hold the sagittal rod 52 in a predetermined position with respect to the sagittal housing 52. Alternatively, the projection can be formed on the sagittal adjustment means 53 and the recess formed on the sagittal rod 52.

In an alternative to this embodiment that is entirely envisageable, the second adjustment means 38 can be arranged at the level of the anterior transverse portion 22 and/or at the level of the posterior transverse portion 24, at least one of the two transverse portions 22, 24 being linked to the upper support 30 via a frontal portion. The adjustment means 38 is then located at the junction of the transverse portion 22, 24 linked to the upper support 30, and the frontal portion, so as to render the upper support 30 mobile in the sagittal plane.

In this embodiment, the adjustment means 38 can also comprise an adjustment means, which can be a button that can be displaced in rotation about itself, providing a degree of freedom at the level of the junction of the frontal portion and the transverse portion 22, 24 linked to the upper support 30, so as to render the upper support 30, bearing at least one electrode, mobile in the sagittal plane.

According to a variant of this alternative, the adjustment means can define pre -established adjustment positions of the upper support 30 in the sagittal plane of the individual. Optionally, according to this alternative embodiment, it is possible for the anterior 14 and posterior 16 supports not to be necessarily present.

In addition, the acquisition device 10 comprises a third adjustment means 40 in order to allow an adjustment of the position of the first 32, second 34 and third 33 acquisition electrodes in the transverse plane Pt. The third adjustment means 40 allows the anterior 22 and posterior 24 transverse portions to be displaced with respect to one another in a direction comprised within the transverse plane Pt. Parallel to the positioning of the first 32, second 34 and third 33 acquisition electrodes in the transverse plane Pt, the third adjustment means 40 also makes it possible to adapt the acquisition device 10 to the head of the user 12 in the transverse plane Pt, i.e. to the circumference of the head of the user 12. With reference to FIG. 4, the third adjustment means 40 comprises a first transverse rod 54 and a second transverse rod (not shown) formed by the anterior support 14 on either side of the head of the user 12. The third adjustment means 40 also comprises a first and a second transverse housings (not shown) formed in the posterior support 16 and in which the first and second transverse rods 54 are capable of sliding, respectively. The acquisition device 10 is formed so that the first transverse rod 54 and the first transverse housing are formed at the level of a first side of the acquisition device 10, here the right-hand side of the head of the user 12, and the second transverse rod and the second transverse housing are formed on a second side of the acquisition device 10 opposite to the first side, here the left-hand side of the head of the user 12.

Similarly to the second adjustment means 38, the third adjustment means 40 can comprise a transverse adjustment means 62 of the position of the first 54 and second transverse rods in order to allow adjustment of the position of the anterior 22 and posterior 24 transverse portions with respect to one another. The transverse adjustment means 62 is then capable of engaging with the first 54 and second transverse rods in order to slide them respectively inside the first and second transverse housings, so as to displace the anterior support 14 with respect to the posterior support 16. The transverse adjustment means 62 adopts the form of a button that can be displaced in rotation about itself in order to slide the first 54 and second transverse rods respectively inside the first and second transverse housings. The adjustment means 62 is here described as a direct adjustment means. As an alternative to a turn button, the transverse adjustment means 62 can be of any form making it possible to slide the first 54 and second transverse rods inside the first and second transverse housings. In addition, as an alternative to combined sliding of the first 54 and second rods, the transverse adjustment means 62 can be configured in order to slide only one of the first 54 and second rods.

According to an alternative embodiment, the adjustment making it possible to slide the first 54 and second transverse rods inside the first and second transverse housings can be carried out manually for example, by the user 12 simply exerting pressure. Thus, the first 54 and second transverse rods then penetrate inside the first and second transverse housings, extending both at the level of each of the bearing portions 46 and each side of the posterior transverse portion 24. In a second phase, the transverse adjustment means 62 can be used to continue the movement previously initiated manually. Alternatively, still in a second phase, the transverse adjustment means 62 may optionally be used only to hold the first and second transverse rods in a predetermined position inside the first and second transverse housings. In the latter case, the adjustment means 62 is described as an indirect adjustment means.

In order to hold the first 54 and second transverse rods in a predetermined position with respect to the first and second transverse housings, the third adjustment means 40 can comprise at least one projection formed on the first 54 and second transverse rods and at least one recess formed on the transverse adjustment means 62. The projection and the recess are capable of engaging with one another in order to hold the first 54 and second transverse rods in a predetermined position with respect to the first and second transverse housings. Alternatively, the projection can be formed on the transverse adjustment means 62 and the recess formed on the sagittal rod 52. In order to facilitate repeated use of the acquisition device 10 on one and the same user 12, the first 36, second 38 and third 40 adjustment means can comprise a graduation or any indicator making it possible to determine the adjustment positions of the first 36, second 38 and third 40 adjustment means. The adjustment positions are for example an angular position of a button, a linear position of a slider or also a relative position of a rod with respect to a housing in which the rod slides.

To this end, the ease of repeatability of the positioning of the first 32, second 34 and third 33 acquisition electrodes can also be permitted by the fact that, according to a particular embodiment of the invention, the upper support 30 on which the first 32, second 34 and third 33 acquisition electrodes are directly or indirectly arranged is capable of being arranged equidistantly from the anterior transverse portion 22 and the posterior transverse portion 24. Thus the adjustment means 53 makes it possible to move the anterior transverse portion 22 closer to or further from the posterior transverse portion 24 equidistantly with respect to the upper support 30 on which the first 32, second 34 and third 33 acquisition electrodes are directly or indirectly arranged. This movement closer or further is provided by means of nesting the anterior sagittal portion 18 of the anterior support 14 within the posterior sagittal portion 20 of the posterior support 16. This positioning of the upper support 30 equidistantly from the anterior transverse portion 22 and the posterior transverse portion 24 has the advantage of allowing placement of the first 32, second 34 and third 33 acquisition electrodes substantially facing the positions C3, C4 and Cz respectively.

In order to improve the hold of the acquisition device 10 on the head of the user 12, the acquisition device 10 also comprises a bearing portion 46 configured in order to bear on an ear of the user 12 when the acquisition device 10 is arranged on the head of the user 12. The bearing portion 46 is formed at the level of the anterior support 14. In order to further improve this hold, the acquisition device 10 comprises another bearing portion 46 making it possible to rest the acquisition device 10 on both ears of the user 12. In addition, the bearing portion 46 makes it possible to provide a reference position for positioning the anterior support 14, which makes it possible to facilitate the satisfactory positioning of the first 32, second 34 and third 33 acquisition electrodes at positions C3 and C4, respectively, of the user 12. Moreover, by means of the configuration thereof allowing it to bear on one ear of the user 12, the bearing portion 46 can also allow optimum positioning of at least one of the reference or passive electrodes facing at least one of the mastoid processes of the user.

In order to facilitate the positioning of the posterior support 16, the acquisition device 10 also comprises a hollow 48 capable of indicating a reference position of the acquisition device 10. The hollow 48 is formed in the posterior support 16 so as to be arranged facing the external occipital protuberance, preferably facing the inion situated at the level of the external occipital protuberance of the head of the user 12, which makes it possible for the arched portion 11 to extend in the sagittal plane Ps. Alternatively, the hollow 48 can adopt any other form making it possible for the acquisition device 10 to have a reference position in order to position the first 32, second 34 and third 33 acquisition electrodes at the positions C3, C4 and Cz, respectively, of the user 12.

The bearing portion 46 and the hollow 48 allow positioning of the posterior 16 and anterior 14 supports adapted to the morphology of the user 12 guaranteeing satisfactory positioning of the first 32, second 34 and third 33 acquisition electrodes in the sagittal Ps and transverse Pt planes. Alternatively, the bearing portion 46 can be formed at the level of the posterior support 16 and the hollow 48, or more generally an indicator of a reference position, can be formed at the level of the anterior support 14 in order to also guarantee positioning of the anterior 14 and posterior 16 supports suitable for the morphology of the user 12.

In addition, the acquisition device 10 comprises a processing unit (not shown) connected to the first 32, second 34 and third 33 acquisition electrodes and, if applicable, to the passive and reference electrodes in order to process information transmitted by these electrodes. The processing unit is housed inside the posterior support 16 and connected to the first 32, second 34 and third 33 acquisition electrodes and, if applicable, to the passive and reference electrodes.

In order to allow transmission outside the acquisition device 10, the acquisition device 10 also comprises a unit for the transmission of the information processed by the processing unit. The transmission unit comprises a port 64 mounted on the posterior support 16 in order to allow wired transmission. Alternatively, the transmission unit can be configured in order to allow transmission of the information via any wireless means, for example via WiFi, Bluetooth, infra-red, or also radio waves.

In order to allow a power supply to the processing unit, the transmission unit and the different electrodes, the acquisition device 10 also comprises a battery, for example a rechargeable battery. Alternatively or in addition to the battery, the acquisition device 10 can be configured in order to be connected to the grid. The acquisition device also comprises an on-off switch 66 of the acquisition device 10.

According to a preferred embodiment of the invention, the acquisition device 10 comprises visual indicators of the state of charge of the battery 68 and of the presence of a connection 70 between the transmission unit and a device for receiving information external to the acquisition device 10. This external device can be a mobile phone connected to the transmission unit. In this case, the processing unit is configured in order to transmit to the mobile phone information relating to the quality of the physical contact between each of the first 32, second 34 and third 33 acquisition electrodes and the scalp of the user, but also in order to subsequently allow the mobile phone to emit a warning signal reporting said quality of the physical contact established.

According to an alternative embodiment of the invention, in order to advise the user 12 of the satisfactory positioning of the first 32, second 34 and third 33 acquisition electrodes, i.e. at the positions C3, C4 and Cz respectively, the processing unit can be configured in order to emit a warning signal. This warning signal can be audible or visual. Additionally or alternatively, the processing unit can be suitable for emitting any other type of warning signal and in particular, tactile, olfactory or also gustatory. Emission of a warning signal by a processing unit incorporated into the acquisition device 10 allows said acquisition device 10 to be independent with respect to the positioning of the electrodes, since there is no need for an external appliance in order to arrange the electrodes in their desired position.

The acquisition device 10 can be positioned on the head of the user 12 by means of the method described hereinafter. The anterior support 14 is firstly displaced with respect to the posterior support 16 so that the annular portion 13 has a circumference equal to or greater than the maximum circumference of the head of the user 12, considered in the transverse plane Pt. In other words, the third adjustment means 40 is actuated so that the acquisition device 10 can be slipped onto the head of the user 12. Then, the acquisition device 10 is positioned on the head of the user 12 so that the bearing portion 46 bears on an ear of the user 12, and the hollow 48 faces the external occipital protuberance of the user 12. The third adjustment means 40 is actuated again so that the annular portion 13 grips the head of the user 12 in the transverse plane Pt. Once this adjustment has taken place, the adjustment means 62 can be actuated so as to lock the annular portion 13 in a final position considered optimum, tightly fitting around the head of the user 12 in the transverse plane Pt. The second adjustment means 38 is actuated so that the arched portion 11 tightly fits around the head of the user 12 in the sagittal plane Ps. The first adjustment means 36 is then actuated in order to position the first 32, second 34 and third 33 acquisition electrodes in the frontal plane Pf. Alternatively, the first adjustment means 36 can be actuated before, or in parallel with, any step of the method described above.

In order to simplify the positioning of the first 32, second 34 and third 33 acquisition electrodes, the positioning method can also comprise determining first and second adjustment positions of the first 36 and second 38 adjustment means respectively. The first and second adjustment positions are then memorized in order to subsequently allow reuse thereof.

The acquisition device 10 is removed from the head of the user and deadjusted, for example as a result of use of the acquisition device 10 on another user 12. By “deadjusted” is meant the fact that the first and second adjustment positions are different from the memorized first and second adjustment positions.

The acquisition device 10 is then replaced on the head of the user 12, but actuating the first 36 and second 38 adjustment means so as to arrange them in the memorized first and second adjustment positions. Positioning the first 32, second 34 and third 33 acquisition electrodes at the positions C3, C4 and Cz is thus facilitated and accelerated. Determining or quantifying the adjustment positions is made possible because the adjustment of the position of the first 32, second 34 and third acquisition electrodes is separate for each of the sagittal Ps, frontal Pf and transverse Pt planes.

Furthermore, the acquisition device 10 can be used among other things in a method for characterizing the sleep of the user 12 presented hereinafter.

In the sleeping state, the difference in electric potential between the first 32, second 34 and third 33 acquisition electrodes and, if applicable, the reference and passive electrodes is measured in order to obtain the brain activity signal representative of the brain waves of the user 12. This brain activity signal is amplified and digitized by the processing unit.

In the frequency range from 9 to 16 Hz of the brain activity signal in the light sleep phase (phase called stage N2 of non-rapid eye movement (NREM) sleep) (De Gennaro & Ferrara, 2003; De Gennaro, Ferrara, Curcio, & Cristiani, 2001), the processing unit identifies the reduced range of sleep spindles of the user 12. This reduced range of sleep spindles comprises the frequencies of brain waves of the sleeping user having an amplitude greater than 15 μV and a period comprised between 0.5 second et 2 seconds. In the awake state, the difference in electric potential between the first 32, second 34 and third 33 acquisition electrodes and, if applicable, the reference and passive electrodes is measured by the acquisition device 10 in order to obtain the brain activity signal representative of the brain waves of the user 12. This brain activity signal is amplified and digitized by the processing unit.

In the frequency range corresponding to the reduced range of sleep spindles, the processing unit compares at least one of the following two parameters, which are the amplitude or the density, to a threshold.

A warning signal is emitted when the parameter of the brain activity signal in the frequency range corresponding to the reduced range of sleep spindles reaches or exceeds the threshold. This warning signal can be audible or visual. Additionally or alternatively, the processing unit can be suitable for emitting, directly or via an external device, any other type of warning signal and in particular, tactile, olfactory or also gustatory.

The user 12 is thus advised of the emission of brain waves promoting the quality of sleep.

On the basis of this characterization of the sleep, a neurofeedback treatment can be put in place to improve the quality of the sleep of the user 12. By neurofeedback is meant a technology making it possible to lead an individual to act on a biological activity, here nervous or neurological, by means of a feedback treatment. This neurofeedback treatment for example consists of training the individual to help him to relax, and prompt him to modify his brain activity. The neurofeedback treatment then comprises training aimed at prompting the user 12 to produce the desired brain waves. The training comprises one or more relaxation sequences and exercises rewarding the emission of the desired brain waves by particular warning signals.

During the training, the threshold can be increased to cause the user 12 to progress in producing suitable brain waves. During the training, the threshold can also be lowered to facilitate the task of a user 12 who may be performing less well.

Of course, the present invention is not limited to the examples and the embodiment described and shown, but numerous variants within the reach of a person skilled in the art may be made thereto.

For example, the acquisition device 10 may dispense with the upper support 30. In this case, the arched portion 11 extends in the frontal plane Pf and incorporates the first 32, second 34 and third 33 acquisition electrodes, and the first adjustment means 36.

Alternatively, the upper support 30 can be mounted on the anterior sagittal portion 18 of the anterior support 14 instead of being mounted on the posterior support 16, at the level of the posterior sagittal portion 20. In this case, the bearing portion 46 is formed at the level of the posterior transverse portion 24 and configured so that the anterior transverse portion 22 slides with respect to the bearing portion 46. Thus, the displacement of the anterior transverse portion 22 with respect to the posterior transverse portion 24 allows the adjustment of the position of the first 32, second 34 and third 33 acquisition electrodes in the transverse plane Pt.

In addition, as an alternative to a configuration in which the anterior 14 and posterior 16 supports form both the annular portion 13 and the arched portion 11, the acquisition device 10 can be configured so that the anterior support 14 is suitable for engaging with the posterior support 16 in order to form the annular portion 13 intended to tightly fit around the head of the user 12 in the transverse plane Pt, or capable of engaging with the posterior support 16 in order to tightly fit around the head of the user 12 in the sagittal plane Ps. In these two latter configurations, the acquisition device 10 can comprise additional supports engaging with the anterior 14 and posterior 16 supports in order to hold the acquisition device 10 on the head of the user 12.

Furthermore, as an alternative or addition to the embodiment presented in FIGS. 2 to 4, the acquisition device 10 can provide for other means of adjusting the position of the first 32, second 34 and third 33 acquisition electrodes. For example, the upper support 30 can be displaced with respect to the posterior sagittal portion 20 in a direction comprised within the sagittal plane Ps or can be capable of being displaced in rotation about an axis extending transversally to the transverse plane Pt of the user 12. In addition, the first 42 and second 44 sliders can be capable of being displaced with respect to the upper support 30 in a direction comprised within the sagittal plane Ps so as to be able to adjust the position of the first 32, second 34 and third 33 acquisition electrodes in the sagittal plane Ps and the frontal plane Pf.

In addition, as an alternative to independent actuation of the first 36, second 38 and third 40 adjustment means, only the first 36 and third 40 adjustment means or only the second 38 and third 40 adjustment means can be configured in order to be actuated independently.

The present description is given by way of example and is in no way limitative of the invention. In particular, the invention relates to the adjustment of the position of at least one electrode at the level of at least any one of the positions of the international 10-20 system, and therefore is not limited to the context of the study of sleep disorders or attention deficit disorders with or without hyperactivity (ADHD) with C3, C4 and/or Cz as positions of interest.

It extends for example to the contexts of the study of memory disorders with other positions of interest such as the positions Cpz, FT7, and/or T7, or T8. Additional supports may be required in order to support the electrode(s) in the position(s) thereof, linked directly or indirectly to one and/or other of the posterior 14, anterior 16 and upper 30 supports.

1. An acquisition device intended to be positioned on the head of a user in order to perform an encephalogram, the head of the user being defined in a transverse plane, a sagittal plane and a frontal plane, the device comprising:
at least one acquisition electrode configured in order to be positioned in contact with the head of the user at at least any one of the positions of the international 10-20 system, so as to acquire at least one brain activity signal representative of the brain waves of the user;
at least one first and one second means of adjusting the position of the acquisition electrode in a first and a second plane, respectively;
in which the first and second planes are different and chosen from at least the transverse and sagittal planes of the head of the user,
in which said first and second means of adjusting the position of the acquisition electrode are capable of being actuated independently of one another.

2. The acquisition device according to claim 1, said device also comprising:
a posterior support;
an anterior support capable of engaging with the posterior support in order to form an annular portion intended to tightly fit around the head of the user in the transverse plane and/or capable of cooperating with the posterior support in order to form an arched portion intended to tightly fit around the head of the user in the sagittal plane,
an upper support on which is mounted the acquisition electrode, the upper support being mounted on the anterior or posterior support.

3. The acquisition device according to claim 2, also comprising a third means of adjusting the position of the acquisition electrode, in a third plane chosen from the transverse, sagittal and frontal planes of the head of the user, the first, second and third planes being different, the first, second and third means of adjusting the position of the acquisition electrode preferably being capable of actuation independently of one another.

4. The acquisition device according to claim 3, in which the upper support is combined with the posterior support, in which:
the first adjustment means is capable of displacing the acquisition electrode, with respect to the upper support in a first direction comprised within the frontal plane;
the second adjustment means is capable of displacing the anterior support with respect to the posterior support in a second direction comprised within the sagittal plane; and
the third adjustment means is capable of displacing the anterior support with respect to the posterior support in a third direction comprised within the transverse plane.

5. The acquisition device according to claim 4, in which the first adjustment means comprises a slider capable of sliding in the first direction comprised within the frontal plane in a frontal housing formed in the upper support, the at least one acquisition electrode being mounted on the slider.

6. The acquisition device according to claim 4, in which the second and third adjustment means comprise:
a rod formed on the anterior support;
a housing formed in the posterior support in which the rod is capable of sliding; and
an indirect or direct means of adjusting the position of the rod mounted on the posterior support and capable of cooperating with the rod in order to slide the rod inside the housing so as to displace the anterior support with respect to the posterior support).

7. The acquisition device according to claim 4, in which the second and third adjustment means comprise at least one projection and at least one recess formed on the rod or the direct or indirect adjustment means, respectively, the projection and the recess being capable of cooperating with one another in order to hold the rod in a predetermined position with respect to the housing.

8. The acquisition device according to claim 2, comprising a first, and/or a second and/or a third acquisition electrodes configured in order to be positioned in contact with the head of the user at one of the positions C3, C4 and/or Cz, respectively, of the international 10-20 system, and in which the first, and/or second and/or third acquisition electrodes are mounted on the upper support.

9. The acquisition device according to claim 8, also comprising an acquisition electrode configured in order to be positioned in contact with the head of the user at the position CPz of the international 10-20 system, and/or an acquisition electrode configured in order to be positioned in contact with the head of the user at the position FT7 of the international 10-20 system.

10. The acquisition device according to claim 8, also comprising an acquisition electrode configured in order to be positioned in contact with the head of the user at the position T3 of the international 10-20 system, and/or an acquisition electrode configured in order to be positioned in contact with the head of the user at the position T4 of the international 10-20 system.

11. The acquisition device according to claim 2, in which the anterior support comprises a bearing portion configured in order to bear on an ear of the user when the acquisition device is arranged on the head of the user.

12. The acquisition device according to claim 11, in which the bearing portion comprises at least one reference electrode configured in order to be positioned in contact with the head of the user, preferably facing one of the mastoid processes of the user.

13. The acquisition device according to claim 2, in which the upper support is mounted on the posterior support, the posterior support comprising a hollow capable of indicating a reference position of the posterior support, the hollow preferably being configured in order to be arranged facing the external occipital protuberance, preferably at the inion, of the head of the user.

14. The acquisition device according to claim 2, said device also comprising:
a processing unit connected to the at least one acquisition electrode, and, if appropriate, to the at least one reference electrode in order to process information transmitted by the at least one acquisition electrode and the at least one reference electrode;
a unit for the transmission of the information processed by the processing unit.

15. A method for positioning an acquisition device on the head of a user in order to perform an encephalogram, the head of the user being defined in a transverse plane, a sagittal plane and a frontal plane, the method comprising the steps consisting of:
providing an acquisition device according to claim 3;
arranging the acquisition device on the head of the user;
actuating the third adjustment means in order to tightly fit around the head of the user,
actuating the second adjustment means so as to place the upper support equidistant from a distal end of the anterior transverse portion and a distal end of the posterior transverse portion,
actuating the first adjustment means in order to adjust the position of the at least one acquisition electrode in the frontal plane;
in which the steps of actuating the first and second adjustment means are performed independently.

16. The positioning method according to claim 15, also comprising the steps consisting of:
determining a first and a second adjustment positions of the first and second adjustment means, respectively;
memorizing the first and second adjustment positions;
actuating the first and second adjustment means so as to position them in adjustment positions different from the first and second adjustment positions;
removing the acquisition device from the head of the user;
replacing the acquisition device on the head of the user;
actuating the first and second adjustment means so as to position them according to the first and second adjustment positions.

17. The positioning method according to claim 15, in which the acquisition device is an acquisition device according to claim 4, in which the step consisting of arranging the acquisition device on the head of the user comprises the steps consisting of:
displacing the anterior support with respect to the posterior support so that the arched portion has a circumference equal to or greater than the maximum circumference of the head of the user, considered in the transverse plane;
positioning the acquisition device on the head of the user so that the bearing portion bears on an ear of the user;
positioning the acquisition device on the head of the user so that the hollow of the posterior support is facing the external occipital protuberance of the head of the user.
